(19) United States
(12) Patent Application Publication
(10) Pub. No.: US 2023/0165924 A1
(43) Pub. Date: Jun. 1, 2023

(54) Herbal Formulation for the Prevention and Management of COVID-19 by Regulating Immunomodulatory Properties

(71) Applicants: Khushbu Kumari, Hisar (Haryana) (IN); Guru Prakash, Hisar (Haryana) (IN)

(72) Inventors: Govind Prasad Dubey, Varanasi (IN); Naveen Agrawal, Hisar (Haryana) (IN); Parul Agrawal, Hisar (Haryana) (IN); Kunka Mohanram Ramkumar, Tamil Nadu (IN); Rajappan Chandra Satishkumar, Tamil Nadu (IN); Ram Gopal Saini, Bathinda (IN); Rajesh Dubey, Hisar (Haryana) (IN)

(73) Assignees: Khushbu Kumari, Hisar (Haryana) (IN); Guru Prakash, Hisar (Haryana) (IN)

(21) Appl. No.: 17/681,368

(22) Filed: Feb. 25, 2022

(30) Foreign Application Priority Data
Dec. 1, 2021 (IN) 202111055672

(51) Int. Cl. A61K 36/72; A61K 36/59; A61K 36/53; A61P 31/14; A61K 45/06

(52) U.S. Cl. CPC A61K 36/72; A61K 36/53; A61K 36/59; A61K 45/06; A61P 31/14

(57) ABSTRACT

The present invention relates to a herbal formulation for the prevention and management of COVID-19 comprising hydro-alcoholic and n-hexane extracts of Hippophae rhamnoides, and hydro-alcoholic extracts of Tinospora cordifolia and Occimum sanctum and the bio-molecules of the said plants regulate the ACE2 receptors in different organ system.

This application claims priority benefit from Indian Patent Application No. 202111055672, filed on Dec. 1, 2021, the entire content of which is incorporated herein by reference. All references cited anywhere in this specification are incorporated by reference as if each had been individually incorporated.

### FIELD OF INVENTION

The present invention relates to an herbal formulation for prevention and management of COVID-19 by regulating immunomodulatory property.

A new technology used in process development including mechanism of action and dynamic process to develop new immune-booster formulation. Taking the lead from traditional medical knowledge system already practiced by the tribal population, who have preserved rich traditional practice. We have developed a novel formulation which found to be useful in the prevention and management of COVID-19. It is now globally accepted that India has preserved rich traditional knowledge which is already in practice, but, could not get acceptance in the scientific world due to lack of mechanism-based study.

Recently, several causative factors have been identified which are associated with development of immune system in almost every society including tribal and rural population. Both genetics and environmental factors are associated with COVID-19. The COVID-19 is a novel virus which has affected human being through respiratory system and produces varying initial clinical manifestations like fever, cough and cold, respiratory distress if not treated properly, later it will influence lower gastrointestinal tract (GI) tract, renal function and cardiovascular manifestations.

### BACKGROUND OF INVENTION

Covid-19 viral pandemic spread across continents and afflicted more than 249 million people and more than 5 million deaths worldwide. Disease pathogenesis and high mortality have been attributed to an unprecedented inflammatory process due to Covid-19 viral infection. This virus has the highest infectivity with an Arnott value ~ 3, due to high binding affinity to the surface antigen ACE2 receptor. The high binding affinity allows high percentage of virus to quickly enter the cell and get into the pathogenic pathway for generating huge amount of viral antigen.

It is highly infectious and generate very high level of inflammation creating cascade of reaction leading to multi organ failure and death. This has very high percentage homology to its closest strain, SARS Cov1, yet extreme difference in pathogenesis morbidity and mortality. In terms of intervention from drug, vaccines and immune therapeutics, used for closed relative virus is not being useful for containing this viral pandemic. This opens an entire field for investigation for developing vaccine, therapeutics and novel diagnostics for this virus.

Worldwide efforts in developing drug and vaccine are going on, whereas, in India we have a unique opportunity to use plant based Phytoconstituents, herbal and Ayurvedic medicine of a traditional nature and utilize existing ones as repurposing for novel development of the drug. Inflammation being the main disease pathogenesis and use of immunomodulators, anti-inflammatory and anti-viral from Indian traditional medicine can be of immense use at this point of time.

Coronaviruses (CoVs) are the largest group of viruses belonging to the Nidovirales order, which includes Coronaviridae, Arteriviridae, and Roniviridae families. The Coronavirinae are further subdivided into four groups, the alpha, beta, gamma and delta coronaviruses. This virus particles contain four main structural proteins. These are the spike (S), membrane (M), envelope (E), and nucleocapsid (N) proteins, all of which are encoded within the 3′ end of the viral genome.

The S protein (~150 kDa), utilizes an N-terminal signal sequence to gain access to the ER, and is heavily N-linked glycosylated. In most, but not all, coronaviruses, S is cleaved by a host cell furin-like protease into two separate polypeptides noted S1 and S2. S1 makes up the large receptor-binding domain of the S protein while S2 forms the stalk of the spike molecule.

Novel strain of coronavirus (SARS-CoV-2) has become a source of augmenting morbidity and mortality worldwide for the last years. After active replication within the infected cells (such as airway epithelial cells, endothelial cells, alveolar macrophages and alveolar epithelial cells), SARS-CoV-2 viruses are released and induce the infected cells to undergo pyroptosis. Waning cells releases various virus derived death associated molecular patterns (DAMPs) and pathogen associated molecular patterns (PAMPs) thereby activating the inflammatory cascade in tissue resident macrophages especially alveolar macrophages. Macrophage activation leads to the release of inflammatory cytokines such as Interleukin (IL)-1β and IL-6 that further promotes the recruitment of neutrophils. Moreover, this condition leads to development of “Cytokine Storm Syndrome” by enhanced levels of inflammatory cytokines and chemokines IL-1β, IL-6, tumor necrosis factor (TNF)-α, chemokine (CC) ligand-2, CCL-3 and CXCL10etc. by innate immune cells that further impose multiple-organ failure and eventually leading to death (Wang et al., 2020). Thus, we can infer from the data that an extreme inflammatory immune response is believed to be the major cause of severity and death in COVID-19 infected patients. Moreover, various studies have clearly specified that traditional medicines/herbal drugs exhibit immunomodulatory properties and are known to augment the expression level of anti-inflammatory cytokines along with reducing inflammatory cytokines expression such as IL-6, IL-17 and TNF-α under extreme inflammatory conditions.

Recently, a distinct coronavirus has been identified as the aetiological agent of SARS, an acute pulmonary syndrome characterized by atypical pneumonia that results in progressive respiratory failure and death in close to 10% of cases. Analysis of the SARS-CoV genome suggests that this virus does not belong to any of the three defined coronavirus groups and that the SARS-CoV and S protein is similarly. So far two types of coronavirus surface receptors have been identified. The coronavirus mouse hepatitis virus uses murine carcinoembryonic antigen related cell adhesion molecules, members of the immunoglobulin super-family of receptors. The coronaviruses for human 229E, transmissible gastroenteritis virus, and feline infectious peritonitis virus, require the zinc metalloprotease aminopeptidase-N for entry into their target cells. This disease is an infectious bronchitis virus that is cleaved by a cellular protease in virus producing cells. The S proteins of other corona viruses, including SARS-CoV, are not cleaved in virus-producing cells. These domains of all characterized corona viruses mediate an initial high affinity association with their respective receptors.

A number of antibodies, peptides and small compounds have been shown to bind to ACE2. It is possible that some of these may be useful in the treatment of SARS, either by blocking the S-protein-binding site or by inducing a conformation in ACE2 is unfavorable to binding or fusion. The SARS-CoV strain was passaged on Vero E6 cells. 293T cells transfected in 25 cm² flasks with pcDNA3.1 alone or pcDNA3.1 expressing ACE2 were infected for 1 h with 1.4 ×10³ TCID₅₀ (50% tissue-culture infectious dose) of SARS-CoV, as measured by endpoint titration on Vero E6 cells, or left uninfected, and washed twice in the culture medium. Cells were monitored for cytopathic effect for 4 days. Alternatively, a soluble form of the receptor itself may slow viral replication in an infected individual. Identification of ACE2 as a SARS-CoV receptor will facilitate the description of the receptor-binding domain of the S protein, presumably the most effective target epitope of an S1-protein-based subunit vaccine. Also, it is likely that a cell line approved for vaccine production and made permissive for viral replication by ACE2 expression will be the most efficient large-scale producer of a whole-killed or attenuated virus for use as a vaccine. A mouse transgenic for human ACE2 may be useful as an animal model of SARS. Finally, the study of the interaction between the SARS-CoV S protein and ACE2 of other animals may provide insights into the origins of the virus. Thus, if SARS returns as a threat to human health, these studies may contribute to its control. RNA was recovered using a viral RNA mini-prep kit. Semi-quantitative RT-PCR was performed using a nested protocol. Virus titration was performed by seeding 5×103 Vero E6 cells per well in 96well micro titre plates 1 the day before infection. Culture supernatant from infected 293T cells was added to the first set of wells in triplicate and serially diluted. Cells were monitored for CPE 3 days after infection of Vero E6 cells. The effect of affinity-purified goat anti-ACE1 or -ACE2 antibody on SARS-CoV-induced cytopathicity was measured by reading absorbance at 492 nm of cells incubated with Cell Titer 96.

The present study is to draw the attention of the community medicine, claiming that herbal formulation specially in Hippophae rhamnoides (Seabuckthorn) berry’s lactic acid bacteria can contain the spread of the new coronavirus by inhibiting the activation of its energy source-purine. This study was confirmed by the researcher of Ehwa Womans University Medical Center, Korea it has been confirmed probiotic bacteria extracted from fermented Seabuckthorn berry had lots of Lactobacillus gasseri (L. gasseri), which represses the activation of purine, an energy source required for the mutation of new coronavirus. This study was found L. gasseri while conducting experiments related to the inhibition of cytokine activity, and Escherichia coli that inflame the bladder. The Seabuckthorn berry had abundant amounts of Streptococcus thermophilus and Lactobacillus rhamnosus, which have the same chemical binding site to the COVID-19 and affect the protein activity. They also confirmed nine antioxidants, six minerals, and 16 amino acids it affects the activation of purine enzyme, the energy source for AIDS, hepatitis, Ebola, cystitis and hyperlipidemia including coronavirus. We have found the drug has antiviral activity in cells by confirming its efficacy in clinical practice and expect the drug will help treat coronavirus patients.

### OBJECTS OF THE INVENTION

An object of the present invention is to propose a herbal formulation which is beneficial in the prevention and management of COVID-19 by regulating immune system (both cellular and humeroal immunity).

Another object of the present invention is to provide the plant based formulation containing Seabuckthorn (Hippophae rhamnoides), Tinospora cordifolia and Occimum sanctum for the protection of respiratory tract and other vital organs from the infection of COVID-19.

Yet another object of the present invention to protect the severity of clinical manifestation after receiving the infection of corona virus among aged individual or comorbid with poor immunity.

Still another object of the present invention is to improve immune profile and the immunomodulatory activity i.e. IgG, IgM, IgA, IgE, general blood picture (TC, neutrophils, lymphocytes, eosinophil count, hemoglobin, total serum protein, platelet count, RBC estimation) among aged populations.

Further object to prevent the vital organs like respiratory tract, lungs, cardiovascular system, with one or two co-morbidities like type-2 diabetes and hypertension.

Still further object of the present invention to protect the cardiovascular events among susceptible individuals.

Still another object of the present invention to protect the neurological manifestations particularly dementia, loss of lean body muscle mass, leading to difficulty in movement of the body.

Yet another object of the present invention to protect and prevent the renal failure due to the COVID-19 infection.

Another object of the present invention is to regulate gut microbiota among COVID-19 infections.

Yet another object of the present invention is to minimize the inflammatory cascades particularly expansion of total WBC count, neutrophils, leukocytosis including absolute neutrophil count and D-Dimmer among COVID-19 infected persons.

### STATEMENT OF INVENTION

According to this invention there is provided a herbal formulation for the prevention and management of Covid-19 by slowing down the progression, enhancing the immune system, reducing the pro-inflammatory cytokines and managing the quality of life by reducing anxiety, stress and depression. The said formulation comprises of at least three plants i.e. Hippophae rhamnoides, Tinospora cordifolia and Occimum sanctum.

Further, according to this invention, there is also provided a process for preparation of novel herbal formulation containing n-hexane and hydro-alcoholic extract of Hippophae rhamnoides, hydro-alcoholic extracts of Tinospora cordifolia and Occimum sanctum by using water (aqueous) and alcohol (30:70) at 70-80° C. and maintaining the pH of solution between 7-10, separating chromatographically the active compound by using TLC, HPLC and HPTLC, supporting with molecular characterization of the plant extract by using IR and NMR.

### DETAILED DESCRIPTION OF THE INVENTION

The hydro-alcoholic extract of Tinospora cordifolia and Occimum sanctum was prepared by using 30:70 ratio of water (aqueous) and alcohol. The water utilized for extraction was decontaminated for any type of bacterial or abnormal growth by using reverse osmosis plant. After extraction the active molecules was identified and separated by HPLC, HPTLC and NMR procedure. The extract of the plant Hippophae rhamnoides was prepared using leaf in hydro-alcoholic and fruit pulp using n-hexane.

The biological activity was studied on the basis of mode of action of the test drug and effect on various parameters undertaken for this clinical condition. The molecular characterization was done by using NMR and bio-molecular reaction following the interaction between the chemical and biological markers responsible for oxidative stress, dyslipidemia, hyperglycemia, hyperhomocysteinemia, hyperleptinemia, including inflammatory process.

The pre-clinical toxicological studies were carried out to determine the safety and efficacy profile of individual as well as combined test formulation before going for human use. The mode of action of single and combined formulation was carried out in animal models.

The anti-inflammatory, antioxidant and enhancing immunomodulatory properties of test formulation were established in animal model before using the drug for human consumption. Safety evaluation studies showed that extract of these three plants i.e., Hippophae rhamnoides, Tinospora cordifolia and Occimum sanctum were well tolerated by in vitro and in vivo studies. A high positive correlation was observed among the in vitro and in vivo assays for anti-oxidative properties. In addition, our results support the potent antioxidant activity of ethanolic extract of three plants which adds one more positive attribute to its known pharmacological properties as anti-cancer drug and hence its use in traditional system of medicine. Further, the dose selection was done on the basis of dose of single plant crude powder and extract yield of the plant. Accordingly, we selected dose of extract by considering the crude form dose. The acute toxicity is done in the light of LD₅₀ determination and ten times higher dose than recommended dose, principle. However, we have determined the safety profile in much higher doses than recommended dose.

### Extraction Procedure

Process for the extraction of test formulation containing leaves and fruit pulp of Hippophae rhamnoides (leaf extract from hydro-alcoholic and fruit pulp extract from n-hexane], stem of Tinospora cordifolia and whole plant of Occimum sanctum. The hydro-alcoholic extract (30:70) were utilized for the extraction of active compound found in plants. After extraction of the plants the chromatography separation were carried out by using TLC, HPLC and HPTLC. After identification and separation of the active compound, the molecular characterization was carried out by using IR and NMR. The phyto-estrogen was separated and identification of ratio between the active molecule and the extract were determined.

The extraction was done at the temperature of 70-80° C. The pH of the solution was maintained between 7-10. The following steps were carried out to separate the active compounds.

[image]

[image]

[image]

According to this invention there is provided an Ayurvedic formulation for the prevention and management of COVID-19 by regulating immune-modulation comprising of the following 3 ingredients given as bellow preferably all the three -

| | |
|---|---|
| Plants Name | Part used |
| 1. Hippophae rhamnoides 2. Tinospora cordifolia 3. Ocimum sanctum | Leaves & Fruits Stem Leaves |

Preferably the aforesaid plants are present in the following doses -

| | |
|---|---|
| Name of the plant | Dose |
| 1. Hippophae rhamnoides 2. Tinospora cordifolia 3. Occimum sanctum | 90-800 mg/day 100-500 mg/day 100-400 mg/day |

The formulation may also comprise known additives such as minerals, vitamin, salts filler (for capsulation or to prepare syrup) and binders, if required to present in trace amount. Average Molecular range of Bio-markers of test formulation:

| Hippophae rhamnoides | Molecular range in (%) |
|---|---|
| 1. Quercetin | 1.5 - 2.62 % |
| 2. Omega 3 fatty acid | 1.2 - 2.12 % |
| 3. Vitamin C | 2.5 - 8.60 % |
| 4. Folic acid | 1.7 - 3.80 % |

| Tinospora cordifolia | Molecular range in (%) |
|---|---|
| 1. Tinosporin | 0.19 - 2.22 % |
| 2. Eleutheroside B | 0.10 - 2.35 % |
| 3. Jatrorrhizine | 0.12 - 2.17 % |
| 4. Berberine | 0.25 - 1.23 % |

| Ocimum sanctum | Molecular range in (%) |
|---|---|
| 1. Oleanolic acid | 0.10 - 2.85 % |
| 2. Ursolic acid | 0.13 - 1.20% |
| 3. Carvacrol | 0.17 - 3.25 % |
| 4. β-caryophyllene | 0.20 - 2.10 % |

Thus, any known additives or supplement is added to prepare the final formulation if required and present in trace amount. Reference is made here in capsule form. However, it would be apparent that the preparation may also be in the form of syrup/capsule.

Preferably but without implying any limitation the preparation comprises -

| | |
|---|---|
| Name of the plant | Dose |
| 1. Hippophae rhamnoides 2. Tinospora cordifolia 3. Occimum sanctum | 450 mg/day 350 mg/day 200 mg/day |

The present novel formulation is based on the combined effect of the three plant extract namely Hippophae rhamnoides, Tinospora cordifolia. and Occimum sanctum. Taking lead from Ayurvedic literature, a combined formulation was prepared after determination of pre-clinical safety and efficacy profile on various animal models (stress model - sleep deprivation, immobilization stress) were designed and effect of test formulation was assessed on helper T-cell, β-cells proliferation, WBC, platelet, RBC including immune profile. Dysregulation of immune system is due to over protection of pro-inflammatory cytokines among persons having co-morbidities like type-2 diabetes, hypertension and individual suffering from protein energy malnutrition. The cytokine storm is mainly due to hyperactivity of the genes associated with imbalance of T-cell and B-cell.

The suppression of nk-cell particularly perforin, IL-2 and TNF-α suppresses the markedly decreases the protection of nk-cell with the result there is the imbalance of T-cell and B-cell. Mass cell increases the IL-1β, IL-6 and IL-33 histamine and protease. The granulocyte particularly neutrophil, eosinophil and basophil enhance a number at the result, total WBC count significantly increases. Abnormal increase in WBC count along with increase in neutrophil, eosinophil and basophils represents the severity of the disease due to cytokine storm. SARC-COV-19 enters into the lung cells by ACE-II receptors, this receptor is present in different organs like brain, lung, intestine, liver and kidney. Therefore, the above organs are more susceptible for damage. The droplet infection, first influence the lung cells and circulated through blood stream as a result the organs having ACE-II inhibitor express resulting in varying degree of clinical manifestation. It also effects the coagulation factor as a result the formation of thrombus block the coronary artery disease resulting cardiac arrest. Therefore, respiratory and cardiovascular failure is the major cause of death among SARC COVID-19.

This Study have shown that quercetin is effective in decreasing serum homocysteine level in high methionine-fed rats and one of possible mechanisms is associated with increased transsulfuration of homocysteine. Quercetin acts as a pro-oxidant, thus converting the aglycone to the metabolites may be helpful in avoiding the harmful effects, and the metabolites have been shown to retain their antioxidant properties. Quercetin glucosides are able to pass through the epithelial cell layer, but they have a lower efficiency than the quercetin. Therefore, the hydrolysis of the glucoside to the aglycone accelerates the absorption of quercetin. Quercetin has beneficial effects on liver fibrosis in rats by enhancing antioxidant enzyme activity and decreasing the pro-oxidant effect. Hippophae rhamnoides is a rich source of flavonoids, vitamins, proteins, amino acids, folic acid, phytosterol, alpha-tocopherol and phenolic compounds. There are at least 24 chemical elements present in Seabuckthorn juice e.g., nitrogen, phosphorous, iron, manganese, boron, calcium, aluminum, silicon and others. It has shown antioxidant, immuno-modulatory, anti-inflammatory and homocysteine lowering effects and uplifts the mental function. Tinospora cordifoliais the active constituents are diterpene compounds including tinosporone, tinosporic acid, cordifolisides A to E, syringen, the yellow alkaloid, berberine, Giloin, crude Giloininand, a glucosidal bitter principle as well as polysaccharides, including arabinogalactan polysaccharide (TSP). Picrotene and bergenin are found in the plant. An immunologically active arabinogalactan, Lincardiosife, cardioside, sesquiterpens are present in this plant. The extract of the plant showed the prevention of falls in leukocyte count which may be due to its rasayana effect. Reduction in eosinophil count has been reported with immunotherapy, that support the immunomodulatory action of this plant. Tinospora cordifolia is also capable of stimulating B-lymphocytes, macrophages and polymarphoneuclear leucocytes.

Details regarding the plants:
1. Hippophae rhamnoides: It belongs to family Elaeagnaceae. In India Hippophae rhamnoides is grown in high temperate zone and Western Himalayas particularly Leh, Laddakh, Himachal Pradesh, Uttarakhand and Sikkim. Two species of Hippophae i.e., salcifolia and rhamnoides are found in India. Seabuckthorn berried have attractive colours varying from yellow, orange to red. The fruits are oval extended oval, round oval in shape. It includes with the succulent calyx tube, drupe like carpodermis membranous or thin leathery, single seed with bony seed coat. High altitude plant Hippophae rhamnoides contains biologically active substance that enhances body immunity, beneficial in age related cognitive deficits, improves memory and attention. The oil of Hippophae rhamnoides has unique anti-ageing property and stimulates tissue regulation. It protects the functional integrity of mitochondria from radiation induced oxidative stress. The fruits and leaves of Hippophae rhamnoides contains β-carotene, vitamin E, flavonoides, Vitamin-C, elagic acid, and ferulic acid showing anti-oxidant potential. This plant also protects mitochondrial damage of varying etiology.
2. Tinospora cordifolia: The active constituents are diterpene compounds including tinosporone, tinosporic acid, cordifolisides A to E, syringen, the yellow alkaloid, berberine, Giloin, crude Giloininand, a glucosidal bitter principle as well as polysaccharides, including arabinogalactan polysaccharide (TSP). Picrotene and bergenin are found in the plant. An immunologically active arabinogalactan, Lincardiosife, cardioside, sesquiterpens are present in this plant. The extract of the plant showed the prevention of fall in leukocyte count which may be due to its rasayana effect. Reduction in eosinophil count has been reported with immunotherapy, that support the immunomodulatory action of this plant. Tinospora cordifolia is also capable of stimulating P-lymphocytes, macrophages and polymarphoneuclear leucocytes.
   Ocimum sanctum: It’s belongs to family Lamiaceae is called also tulsi. Tulasī is an aromatic plant which is native throughout the Old-World tropics and widespread as a cultivated plant and an escaped weed. It is grown in houses, temples and gardens. Some of the main chemical constituents of Ocimum sanctum are: Oleanolic acid, Ursolic acid, Rosmarinic acid, Eugenol, Carvacrol, Linalool, and β-caryophyllene. Aqueous extract from leaves showed both humoral and cell mediated immune response in rats and mice, it is an immunomodulator. Ocimum sanctum have been evaluated for its various pharmacological activities. It has been found to have analgesic, anti-inflammatory, bronchial asthma and hypotensionactivity. It is also reported that Ocimum sanctum has antistress, antiulcerogenic, antimicrobial activity, in addition to its effect on cancer cells, radioprotection, immunomodulation, anthelmentic, hypoglycemic, antifertility, hepatoprotective and antiallergy.

### Preclinical Evidence

Safety profile- These plants Hippophae rhamnoides, Tinospora cordifolia and Occimum sanctum have been traditionally used as medicine and nutritional supplement for a long period of time. Our study provides valuable data on the safety profile as per regulatory norms in INDIA and USA with result supporting their safe longer-term use in combination for synergetic effects. The experimental data given in Table 1 & 2 and the clinical study tables 1 to 6 clearly reflects that the combination of the plants in the herbal formulation of the present invention gives an unexpected result in case of Covid-19 patients due to the synergism amongst the plant components. Thus, such a herbal formulation is both novel and inventive.

### EXPERIMENTAL STUDY

TABLE-1

Effect of test formulation on general blood picture in experimental animals following stress

| Groups | WBC (Thousand /cu mm) | Lymphocyte (%) | Nutrophils (%) | Platelet count (lacs/cu mm) | Hb (%) |
|---|---|---|---|---|---|
| Normal (N=6) | 6349.52 ±973.35 | 32.32 ±4.73 | 63.45 ±3.23 | 1.75 ±0.42 | 9.45 ±1.53 |
| Immobilization stress (N=6) | 6539.49 ±923.40 | 30.49 ±3.82 | 59.85 ±2.95 | 1.88 ±0.47 | 8.05 ±1.66 |
| Immobilization stress + Test formulation (N=6) | 6858.75 ±984.54 | 27.22 ±4.38 | 61.93 ±0.85 | 1.58 ±0.43 | 8.92 ±1.74 |

TABLE-2

Results shows the effect of test formulation on immunological parameters following stress in experimental rats.

| Groups | IgG (mg/dl) | | IgA (mg/dl) | | IgM (mg/dl) | |
|---|---|---|---|---|---|---|
| | 7 days | After 15 days | 7 days | After 15 days | 7 days | After 15 days |
| Normal (N=6) | 1088.64 ±139.32 | 1104.73 ±102.55 | 245.73 ±60.42 | 261.34 ±63.08 | 121.62 ±17.09 | 136.09 ±22.65 |
| Immobilization stress (N=6) | 739.42 ±83.60 | 629.73 ±48.54 | 163.68 ±41.62 | 131.90 ±48.01 | 82.57 ±13.80 | 71.35 ±12.83 |
| Immobilization stress + Test formulation (N=6) | 831.64 ±102.34 | 958.39 ±93.68 | 198.42 ±35.64 | 215.68 ±42.75 | 98.45 ±18.36 | 106.38 ±21.98 |

### Clinical Results

TABLE 1

Effect of herbal formulation on immunologic markers among COVID-19 infected patients

| Treatment group | No. of cases | IgG (mg/dl) | | |
|---|---|---|---|---|
| | | Initial | 3 months | 6 months |
| Conventional treatment | 48 | 839.85 ±112.98 | 732.80 ±104.75 | 620.32 ±90.75 |
| Conventional treatment + Test formulation | 52 | 814.85 ±118.76 | 740.32 ±160.28 | 669.41 ±140.55 |

Normal range: 710-1520 (mg/dl)

TABLE 2

Effect of herbal formulation on immunologic markers among COVID-19 infected patients

| Treatment group | No. of cases | IgM (mg/dl) | | |
|---|---|---|---|---|
| | | Initial | 3 months | 6 months |
| Conventional treatment | 48 | 190.50 ±61.30 | 160.55 ±60.32 | 130.64 ±22.80 |
| Conventional treatment + Test formulation | 52 | 200.50 ±88.50 | 180.85 ±24.50 | 160.32 ±25.35 |

Normal range: 40-250(mg/dl)

TABLE 3

Effect of herbal formulation on immunologic markers among COVID-19 infected patients

| Treatment group | No. of cases | IgA (mg/dl) | | |
|---|---|---|---|---|
| | | Initial | 3 months | 6 months |
| Conventional treatment | 48 | 205.37 ±48.30 | 216.85 ±40.32 | 150.60 ±48.32 |
| Conventional treatment + Test formulation | 52 | 199.52 ±22.80 | 167.40 ±20.85 | 168.10 ±17.45 |

Normal range: 90-310 (mg/dl)

TABLE 4

Decrease in Interleukin-6 following herbal formulation treatment in COVID-19 infected patients

| Groups | IL-6 (pg/ml) | | | Comp. Initial vs After 6 months |
|---|---|---|---|---|
| | Initial | After 3 months | After 6 months | |
| Placebo (N=25) | 2.91 ±0.96 | 2.75 ±0.87 | 3.21 ±0.87 | P>0.05 |
| Test formulation (N=33) | 3.14 ±0.98 | 2.76 ±0.83 | 2.15 ±0.72 | P<0.001 |

Normal range: <1(pg/ml)

TABLE 5

Decrease in TNF-α following herbal formulation treatment in COVID-19 infected patients

| Groups | TNF- α (pg/ml) | | | Comp. Initial vs After 6 months |
|---|---|---|---|---|
| | Initial | After 3 months | After 6 months | |
| Placebo (N=25) | 698.65 ±101.84 | 701.36 ±102.38 | 745.61 ±115.87 | P<0.05 |
| Test formulation (N=33) | 729.87 ±126.78 | 583.75 ±91.65 | 532.68 ±82.44 | P<0.001 |

Normal range: 25-800(pg/ml)

TABLE 6

Homocysteine lowering effect of herbal formulation in COVID-19 infected patients

| Groups | H mocystein (mmol/L) | | | Comp. Initial vs After 6 months |
|---|---|---|---|---|
| | Initial | After 3 months | After 6 months | |
| Placebo (N=25) | 26.69 ±3.84 | 26.82 ±4.21 | 25.88 ±4.01 | P>0.01 |
| Test formulation (N=33) | 28.34 ±4.12 | 24.81 ±2.96 | 21.55 ±3.36 | P<0.01 |

Normal range: 5-15(mmol/L)

### EXAMPLES

### Example-I

When the hexane extract of Hippophae rhamnoides in the dose of 75 mg/kg/day and hydro-alcoholic extract of Tinospora cordifolia in the dose of 60 mg/kg/day was given orally to albino rats there is an increase in immune profile and thus prevents the spread of infection among the animals.

### Example-II

When the hydro-alcoholic extract of Tinospora cordifolia in the dose of 65 mg/kg/day and Occimum sanctum in the dose of 40 mg/kg/day was given orally to the experimental model for observing the effect on reducing pro-inflammatory markers like TNF-α, IL-6, IL-1β and γinterferon.

### Example-III

When the hydro-alcoholic extract of Tinospora cordifolia in the dose of 40 mg/kg/day and Occimum sanctumin the dose of 30 mg/kg/day was orally administered to the animal models showed significant reduction in the inflammatory markers like hs-CRP and etherocyte segmentation rate.

### Example-IV

When the extract of Hippophae rhamnoides (60 mg/day) and Tinospora cordifolia (35 mg/day) given to experimental animal models showing significant reduction in atherothrombotic plaques including reduction in elevated platelet counts.

### Example-V

In clinical study when the extract of Hippophae rhamnoides (400 mg/day) and Tinospora cordifolia (250 mg/day) showed beneficial role in reducing proinflammatory cytokines storms particularly TNF-α and IL-6.

### Example-VI

When the extract of Hippophae rhamnoides (350 mg/day) and Occimum sanctum (200 mg/day) given to subjects showing beneficial effect on BMI and minimize the effect of muscular atrophy.

### Example-VII

When the extract of Hippophae rhamnoides (350 mg/day) and Tinospora cordifolia (300 mg/day) and Occimum sanctum (250 mg/day) given to cases showing high glycemic index with elevated inflammatory markers like hs-CRP showed significant reduction in inflammatory markers.

### Example-VIII

When the extract of Hippophae rhamnoides (250 mg/day), Tinospora cordifolia (175 mg/day) and Occimum sanctum (125 mg/day) given to the COVID infected patients showing the evidence of hyperhomocysteinemia showed significant decrease in the homocysteine level in both sex group.

### Example-IX

The extract of Hippophae rhamnoides (300 mg/day) and Occimum sanctum (200 mg/day) given to subjects showing evidence of neurological manifestations exhibited marked improvement in the mental functions.

### Example-X

The extract of Hippophae rhamnoides (350 mg/day), Tinospora cordifolia (250 mg/day) and Occimum sanctum (275 mg/day) given to patients suffering from COVID-19 reduces the oxidative stress particularly lipid peroxidase, catalase, superoxide dismutase, glutathione activity etc. This formulation established the antioxidant role of the drug. The formulation also exerted immunomodulatory property.

### Example-XI

When the combined test formulation was given in the dose 500 mg twice a day for the period of 6 months to one year in the prevent the post COVID complications in both sex group particularly general feeling of well-being and restoration of normal organs function.

### Example-XII

When the herbal extract of Hippophae rhamnoides (350 mg/day) and Tinospora cordifolia (250 mg/day) and Occimum sanctum (200 mg/day) were administered to cases showing involvement with various risk factors exerted better results. The test drug exerted antiproliferative, hypolipidemic, anti-atherogenic, antioxidant, anti-anxiety with reducing homocysteine level. Thus, it is proposed to be a potent remedial measure for the prevention/minimize the adverse event due to COVID-19.

It is to be noted that the present invention is susceptible to modifications, adaptations and changes by those skilled in the art. Such variant embodiments employing the concepts and features of this invention are intended to be within the scope of the present invention which is further set forth in the claims.

We claim:

1. A herbal formulation for the prevention and management of COVID-19 comprising n-hexane and hydro-alcoholic extracts of Hippophae rhamnoides, and hydro-alcoholic extracts of Tinospora cordifolia and Occimum sanctum and the bio-molecules of the said plants regulate the ACE2 receptors in different organ system.

2. The herbal formulation of claim 1 comprising the plant extracts in amounts to provide the doses:

| Hippophae rhamnoides | 90 - 800 mg/day |
|---|---|
| Tinospora cordifolia | 100 -500 mg/day and |
| Occimum sanctum | 100 -400 mg/day |

and optionally comprising minerals, vitamin, salts, binders, or other known additives.

3. The herbal formulation as claimed in claim 1 wherein the Hippophae rhamnoides (Seabuckthorn) when orally administered in the patients suffering from COVID-19 the provides quercetin, omega-3 fatty acid, folic acid and vitamin-C in an amount effective to enhance body immunity to a subject suffering from COVID-19infection.

4. The herbal formulation as claimed in claim 1, wherein the stem of the Tinospora cordifolia contain tinosporin, in an amount effective to reduce the level of proinflammatory cytokines reduce the clinical manifestation of cytokine storms.

5. The herbal formulation as claimed in claim 1, wherein n-hexane extract of Hippophae rhamnoides (Seabuckthorn) is present in an amount effective to prevent the damage of organ systems of COVID-19 patients.

6. The herbal formulation as claimed in claim 1, wherein the Seabukthorn (Hippophae rhamnoides) causes a reduction in the glycemic index, and a reduction in the level of pro-inflammatory cytokines like IL-6 and TNF-α.

7. The herbal formulation as claimed in claim 1, wherein the formulation is orally administered and reduces amount of an inflammatory marker and prevents inflammation in vital organs.

8. The herbal formulation of claim 7, wherein the inflammatory marker is hs-CRP.

9. A method of treating a subject regulating suffering from COVID-19, comprising orally administering the herbal formulation of claim 1.

10. The method of claim 9, wherein treating comprises regulating abnormal cardiac events in the subject.

11. A method of claim 9, wherein the subject shows evidence of renal dysfunction and treating comprises stabilizing renal function.

12. The method of claim 11, wherein the evidence of renal dysfunction comprises proteinuria, an elevated level of serum creatinine, an elevated level of blood urea or a combination thereof.

13. The method of claim 9, wherein treating comprises enhancing the cellular immunity in the subject as measured by an increase in the level of CD4 and CD8.

14. The method of claim 9, wherein treating comprises increasing the humeral immunity of IGg, IgM, and/or IgA in the subject.

15. The method of claim 9, wherein treating comprises preventing cardiovascular and neurological events in the subject by reducing the level of homocysteine level.

16. The method of claim 9, wherein treating comprises reducing depression in the subject by regulating brain biogenic amines.

17. A process for preparing a herbal formulation comprising
preparing n-hexane and hydro-alcoholic extracts of Hippophae rhamnoides, and hydro-alcoholic extracts of Tinospora cordifolia and Occimum sanctum using water and alcohol in a ratio of 30:70, at a pH between 7-10 and a temperature at 70 -80 C,
separating the solution using chromatography, and
characterizing the extract using NMR and/or IR.
